(19) United States
(12) Patent Application Publication
(10) Pub. No.: US 2017/0296396 A1
(43) Pub. Date: Oct. 19, 2017

(54) ABSORBENT ARTICLE MANUFACTURING PROCESS INCORPORATING IN SITU PROCESS SENSORS

(71) Applicant: The Procter & Gamble Company, Cincinnati, OH (US)

(72) Inventors: Eric Daniel RICCIARDI, Cincinnati, OH (US); Mathias Johannes HILPERT, Mason, OH (US)

(21) Appl. No.: 15/447,721

(22) Filed: Mar. 2, 2017

Related U.S. Application Data
(60) Provisional application No. 62/322,461, filed on Apr. 14, 2016.

(51) Int. Cl. A61F 13/15

(52) U.S. Cl. CPC A61F 13/15772; A61F 13/15699

(57) ABSTRACT

A method for method for fabricating an absorbent article is disclosed. The absorbent article comprises at least a topsheet and a liquid impermeable backsheet. The method provides for the steps of: a) supplying the topsheet; b) supplying the liquid impermeable backsheet; c) affixing a measuring device to one of the topsheet and liquid impermeable backsheet; and, d) contactingly engaging the topsheet and liquid impermeable backsheet so that the measuring device is disposed therebetween when the topsheet and liquid impermeable backsheet are disposed in contacting engagement.

### FIELD OF THE INVENTION

The present disclosure relates to methods for fabricating absorbent articles. Specifically, the method can be used to measure as well as monitor process conditions and web material characteristics during the fabrication of absorbent articles.

### BACKGROUND OF THE INVENTION

Absorbent articles such as sanitary napkins, pantiliners, disposable diapers, incontinence products, and bandages are designed to absorb and retain liquid and other discharges from the human body and to prevent body and clothing soiling.

The different materials forming the absorbent articles are usually supplied as rolls that are continuously unwound and converted to form the various layers of the absorbent article. Such layers normally include at least a topsheet, an absorbent core and a back sheet, and commonly intermediate or additional layers such as a secondary topsheet, secondary backsheet, a layer of adhesive placed on the backsheet to adhere the articles within the undergarment, and the like.

Although the aforementioned processes can produce suitable absorbent articles, it has been found that the absorbent article manufacturing environment has a significant number of process variables that can severely impact the formation of the absorbent article, the performance of the formed absorbent article, and the consumer acceptance of the formed absorbent article.

This could be attributed to the inability to measure certain key physical parameters of the absorbent article manufacturing process unit operations during use and key physical parameters of the absorbent article that impact in-use performance. By way of example, the equipment used in the manufacture of absorbent articles can subject the materials that form the resulting absorbent article to extreme temperatures, bending moments, pressures, tensions, stress, strain, pH, wear, and the like. Each of these factors has been found to have an effect on the construction and/or performance of the absorbent article.

Additionally, the decay and/or failures of materials used to manufacture absorbent articles can also have serious implications on the efficiency of the absorbent article manufacturing process. A high frequency of material failures can substantially affect the economies of an absorbent article business due to the loss of the use of the expensive converting machinery (that is, the machine “downtime”) during the time an unbroken material is being fitted on the converter.

In current assembled products processes (such as the absorbent articles mentioned supra), the materials used to manufacture the absorbent article are the ingredients and pressures, heat, and tensions that drive the converting transformations that turn those ingredients into winning products. However, current manufacturing processes provides for process measurements to come from sensors disposed upon the manufacturing equipment. These process measurements can be provided by non-contact measurement systems. For example, temperature plays an important role as an indicator of the condition of a product or piece of machinery, both in manufacturing and in quality control. Accurate temperature monitoring improves product quality and increases productivity. Downtimes are decreased, since the manufacturing processes can proceed without interruption and under optimal conditions. It is believed that non-contact temperature measurement can provide advantages of measurement speed, facilitating the temperature measurement of moving targets, and/or reducing any risk of product contamination and mechanical effect on the surface of the materials used to manufacture the absorbent articles.

However, the materials used to manufacture the absorbent articles must be optically (infrared-optically) visible to the non-contact temperature measurement system. Any level of dust or smoke can make measurement less accurate. Further, only topical measurements of the materials used to manufacture the absorbent articles can be measured. Further, the differing emissivities of the various materials used to manufacture the absorbent articles must be taken into account. Finally, the optics of the sensor must be protected from dust and condensing liquids. Additionally, the pressures experienced by the materials used to manufacture the absorbent articles in process nips (formed between pressure rolls) and/or vacuum slots, as well as process abrasion points (e.g., while traversing vacuum boxes and the like) and stresses introduced by misaligned process equipment have been found to have an effect on the construction and/or performance of the absorbent article. Current efforts to measure pressures in process nips are generally limited to industrial rolls having a substantially cylindrical core having an outer surface and a polymeric cover circumferentially overlying the core outer surface. The cover generally includes a base layer circumferentially overlying the core, a top-stock layer overlying the base layer, and a sensing system. The sensing system generally provides a material which responds to physical forcing stimuli and changes its electrical properties, providing an analog signal. One of skill in the art will understand that this could include a plurality of piezo-electric, piezo-resistive, piezo-capacitive, and/or combinations thereof sensors embedded in the cover base layer. In any regard, the sensors can be configured to sense pressure experienced by the roll and provide signals related to the pressure, stress, strain, and the like.

Other methods for measuring pressures in process nips (i.e., determine the pressure distribution between mating surfaces) provides for the placement of a thin (e.g., 20 mils) pressure indicating sensor film. The pressure indicating sensor film is placed between two contacting or impacting surfaces. After removal, the resulting image will show the relative amount of pressure applied as a grayscale pressure distribution profile. A greater pressure provides for a darker color intensity. Grayscale pressure distribution profiles can be rendered into interpretable images that can be presented as a high resolution full-color representation of pressure distribution. Unfortunately, pressure indicating sensor films are really only suitable for loads of less than 20,000 PSI (1,500 kg/cm²).

However, these sensors only provide an indication of the pressure exerted upon the materials used to manufacture the absorbent articles during the time the materials used to manufacture the absorbent articles are in contacting engagement with the roll because the measured forces must be extrapolated. There is no effective way to measure the pressure(s) experienced by the materials used to manufacture the absorbent articles when it is not in contacting engagement with the roll.

Further, because absorbent article manufacturing process can have many different performance demands, varying the material employed in the cover can provide the roll with different performance characteristics as the absorbent article manufacturing process demands. However, a particular cover will need to necessarily be provided with a single performance characteristic. This means that if the desired performance characteristic changes, the roll cover, or even the entire roll, must be changed as well as the roll set-up height, pattern, and pressures exerted by the rolls.

Additionally, the polymeric materials for covers typically used (i.e., natural rubber, synthetic rubbers such as neoprene, styrene-butadiene (SBR), nitrile rubber, chlorosulfonated polyethylene (“CSPE”—also known under the trade name HYPALON® from DuPont), EDPM (the name given to an ethylene-propylene terpolymer formed of ethylene-propylene diene monomer), polyurethane, thermoset composites, and thermoplastic composites) may be incompatible with the web materials used for the manufacture of the absorbent articles contemplated herein.

Further, the layers used to provide the cover with a prescribed set of physical properties for operation (e.g., strength, elastic modulus, and resistance to elevated temperature, and the like) must be designed to have a predetermined surface hardness that is appropriate for the process they are to perform, provide for the web material to “release” from the cover without damage to the web material, and be abrasion- and wear-resistant.

However, it can be difficult under certain circumstances to produce and receive consistent signals given the thickness of the covers and the sensitivity of the fiber optic sensors and the optical fibers running to the sensors. Also, the optical fibers routed between the sensors can be brittle, so placement of them in a cover during manufacture can be difficult. Further, the placement of temperature sensors in a roll cover are not a suitable solution since the roll covers tend to heat up during contacting and rotating engagement. In addition, electrical sensors positioned on the core of the roll (beneath the base layer of the cover) typically require electrical insulation and can cause failure of the core-cover bond, which failure can be catastrophic for the cover. In contrast, sensors positioned on top of the base are sufficiently insulated, but are subject to malfunction due to water permeation in the topstock of the cover. Some piezoelectric sensors have been proposed, but many of these have been unsuitable due to their inability to function reliably in the desired temperature range (i.e., the temperature above which proposed piezoelectric materials lose reliable piezoelectric behavior, also known as the Curie temperature, has been too low). In short, such rolls are too inflexible and difficult to install in typical converting process to be useful since they are large, linear systems.

The significance of the difficulties experienced by the manufacturers of these absorbent articles can be exacerbatingly increased by the relatively high cost of the materials used to manufacture the absorbent articles themselves.

Therefore, a need exists for a method of making an absorbent article, and an ability to monitor the physical condition of the materials during use in the production of absorbent articles that can eliminate the foregoing problems. In short, the ability to measure the physical condition of the materials used to manufacture the absorbent articles made by the prior processes during use can provide for real-time in situ feedback into the absorbent article manufacturing process that can stimulate process changes necessary to produce quality absorbent articles and simultaneously increase converting equipment down-time.

In short, there is a need to provide a process that incorporates a sensor that can be dispatched through a converter in order to obtain true in-situ process measurements and understand the physics of transformations like never before. And by combing this sensing technology with an automated data collection system operably affiliated with a converter, these in-situ measurements can be readied for use as real time process controls.

### SUMMARY OF THE INVENTION

The present disclosure is directed to a method for fabricating an absorbent article. The absorbent article comprises at least a topsheet and a liquid impermeable backsheet. The method comprises the steps of: a) supplying the topsheet; b) supplying the liquid impermeable backsheet; c) affixing a measuring device to one of the topsheet and liquid impermeable backsheet; and, d) contactingly engaging the topsheet and liquid impermeable backsheet so that the measuring device is disposed therebetween when the topsheet and liquid impermeable backsheet are disposed in contacting engagement.

The present disclosure is also directed to a method for adjusting a process for manufacturing absorbent articles. The process has a machine direction (MD) and a cross-machine direction (CD) coplanar and orthogonal thereto. The process improves the manufacture of absorbent articles manufactured thereby. The process comprises the steps of: a) providing an absorbent article converter, the absorbent article converter having at least one process set-point, the at least one process set-point being related to at least a first physical characteristic of the absorbent articles; b) providing a first web material associated with the absorbent articles integral with the absorbent article converter; c) attaching a measuring device upon a surface of the first web material, the measuring device being disposed integral thereupon; d) providing a second web material associated with the absorbent articles integral with said converter; e) contactingly engaging the first and second web materials with the converter to provide a contactingly engaged first and second web materials so that the measuring device is disposed between the first and second web materials; f) causing the contactingly engaged first and second web materials to traverse past a receiver, the receiver being in wirelessly communicating engagement with the measuring device when the measuring device is proximate the receiver, the measuring device being capable of wirelessly transmitting information to the receiver, the information comprising data relating to a measurement of the at least one physical characteristic of the contactingly engaged first and second web materials; and, g) changing the process set-point according to the measurement of the at least one physical characteristic of the contactingly engaged first and second web materials.

### BRIEF DESCRIPTION OF THE DRAWINGS

FIG. 1 is a schematic representation of a process for forming absorbent articles having measurement devices attached thereto and reading devices associated therewith for measuring web material physical properties and absorbent article manufacturing process conditions during the formation of absorbent articles of the present disclosure;

FIG. 2 is a schematic representation of another embodiment of a process for forming absorbent articles having measurement devices attached thereto and reading devices associated therewith for measuring web material physical properties and absorbent article manufacturing process conditions during the formation of absorbent articles; and,

FIG. 3 is schematic representation of another means of forming absorbent articles having measurement devices attached thereto and reading devices cooperatively associated therewith for measuring web material physical properties and absorbent article manufacturing process conditions during the formation of first and second discrete features during the formation of an absorbent article before and after matingly engaging two web materials in a face-to-face relationship.

### DETAILED DESCRIPTION OF THE INVENTION

In converting, the term “machine direction” (MD) refers to that direction parallel to the flow of a web material through a converting equipment process.

The “cross-machine direction” (CD) is orthogonal to the MD and in the plane generally defined by the web material.

The “Z-direction” is the direction orthogonal to both the MD and CD.

The term “absorbent article”, as used herein, includes disposable articles such as sanitary napkins, panty liners, diapers, adult incontinence articles, and the like. Such absorbent articles are intended for the absorption of body liquids, such as menses or blood, vaginal discharges, urine, and feces. Various absorbent articles described above will typically comprise a liquid permeable topsheet, a liquid impermeable backsheet joined to the topsheet, and an absorbent core between the topsheet and backsheet.

The term “aperture”, as used herein, refers to a hole. The apertures can either be punched cleanly through the web so that the material surrounding the aperture lies in the same plane as the web prior to the formation of the aperture, or holes formed in which at least some of the material surrounding the opening is pushed out of the plane of the web. In the latter case, the apertures may resemble a protrusion or depression with an aperture therein.

The term “assembled article”, as used herein refers to an article formed from a plurality of materials, or component part thereof, intended to be produced or distributed for sale to a consumer for use in or around a permanent or temporary household or residence, a school, in recreation, or otherwise, or (ii) for the personal use, consumption or enjoyment of a consumer in or around a permanent or temporary household or residence, a school, in recreation, or otherwise. An exemplary assembled article can be formed from a plurality of web materials that when suitably combined to form an absorbent article.

The term “component” of an absorbent article, as used herein, refers to an individual constituent of an absorbent article such as a topsheet, acquisition layer, liquid handling layer, absorbent core or layers of absorbent cores, backsheets, and barriers such as barrier layers and barrier cuffs, and functional or aesthetic design elements such as colored regions, channels, and features formed on a topsheet.

The term “comprising”, as used herein and in the claims, is inclusive or open-ended and does not exclude additional unrecited elements, compositional components, or method steps.

The term “discrete”, as used herein, means distinct or unconnected. When the term “discrete” is used relative to forming elements on a forming member, it is meant that the distal (or radially outwardmost) ends of the forming elements are distinct or unconnected in all directions, including in the machine and cross-machine directions (even though bases of the forming elements may be formed into the same surface of a roll, for example).

The term “forming elements”, as used herein, refers to any elements on the surface of a forming member such as a roll that are capable of deforming a web. The term “forming elements” includes both continuous or non-discrete forming elements such as the ridges and grooves on ring rollers, and discrete forming elements.

The term “joined”, as used herein, refers to the condition where a first component is affixed, or connected, to a second component either directly; or indirectly, where the first component is affixed, or connected, to an intermediate component which in turn is affixed, or connected, to the second component. The joined condition between the first component and the second component is intended to remain for the life of the absorbent article.

The term “nonwoven”, as used herein, refers to a material having a structure of individual fibers or threads which are interlaid, but not in a repeating pattern as in a woven or knitted fabric, which do not typically have randomly oriented fibers. Nonwoven has been formed from many processes, such as, for example, melt-blowing processes, spun-bonding processes, hydro-entangling, and bonded carded web processes, including carded thermal bonding. The constituent fibers of a nonwoven can be polymer fibers, and can be mono-component, bi-component, and/or bi-constituent, and a mixture of different fiber types.

The term “odor control composition”, as used herein, refers to such compositions usually contain, sometimes along with conventional perfume ingredients, ingredients which are able to chemically react with the malodorant molecules released from the body fluids (such as ammonia) thus neutralizing the source of the malodor, and/or ingredients which are able to interact with nose receptors so that their perception of the malodorant molecules is reduced.

The term “phase”, as used herein, refers to the positional relationship between two or more parts of a machine that performs repetitive motion. For example, phase may refer to the relative position of a punch that stamps apertures into a component used in the manufacturing process. When utilized as verbs, the terms “phasing,” “phased,” “phase,” and the like refer to the act of changing the phase of a device from one phase to another. For example, the act of phasing a roller may refer to advancing or retarding the rotation of the roller about its primary axis.

The term “polymer” generally includes, but is not limited to, homopolymers, copolymers, such as for example, block, graft, random and alternating copolymers, terpolymers, etc., and blends and modifications thereof. In addition, unless otherwise specifically limited, the term “polymer” includes all possible geometric configurations of the material. The configurations include, but are not limited to, isotactic, atactic, syndiotactic, and random symmetries.

The term “upper”, as used herein, refers to absorbent members, such as layers, that are nearer to the wearer of the absorbent article during use, i.e. towards the topsheet of an absorbent article; conversely, the term “lower” refers to absorbent members that are further away from the wearer of the absorbent article towards the backsheet.

The term “web material”, as used herein, is intended to refer to any of the materials used in the manufacture of absorbent articles such as those described herein, either individually or collectively (e.g., combined materials). Such materials can include the topsheet material(s), backsheet material(s), precursor sheet(s), precursor topsheet material(s), precursor backsheet material(s), an/or absorbent core material(s), combinations thereof, and the like.

Regarding all numerical ranges disclosed herein, it should be understood that every maximum numerical limitation given throughout this specification includes every lower numerical limitation, as if such lower numerical limitations were expressly written herein. In addition, every minimum numerical limitation given throughout this specification will include every higher numerical limitation, as if such higher numerical limitations were expressly written herein. Further, every numerical range given throughout this specification will include every narrower numerical range that falls within such broader numerical range and will also encompass each individual number within the numerical range, as if such narrower numerical ranges and individual numbers were all expressly written herein.

### Absorbent Article

Assembled articles in the form of absorbent articles can be manufactured by a method according to the present disclosure can generally comprise a liquid permeable deformed topsheet; a liquid permeable colored sheet, and a liquid impermeable backsheet joined to the topsheet, wherein the topsheet comprises a polymer film and a nonwoven, and have a plurality of first discrete features and a plurality of second discrete features; wherein the colored sheet has a first colored region and comprises a precursor sheet; and wherein the backsheet comprises a precursor backsheet.

In one embodiment, the polymer film is a polymer film including materials normally extruded or cast as films such as polyolefins, nylons, polyesters, and the like. Such films can be thermoplastic materials such as low density polyethylene, medium density polyethylene, high density polyethylene, linear low density polyethylene, polypropylenes and copolymers and blends containing substantial fractions of these materials. In another embodiment, the nonwoven constituting the topsheet is a colored nonwoven.

The polymer film can have a plurality of discrete extended elements such as those disclosed in International Patent Application Nos. WO 01/76842, WO 10/104996, WO 10/105122, WO 10/105124, and U.S. Patent Application No. 2012/0277701A1. In one embodiment, the polymer film can have a plurality of discrete extended elements comprising open proximal ends, open or closed distal ends, and sidewalls, wherein the discrete extended elements comprise thinned portions at the distal ends of the discrete extended elements and/or along the sidewalls of the discrete extended elements, and wherein the discrete extended elements have a diameter of less than about 500 microns; the discrete extended elements have an aspect ratio of at least about 0.2; and/or the polymer film comprises at least about 95 discrete extended elements per square centimeter.

In the present disclosure, the colored sheet comprising a precursor sheet may function as a secondary topsheet in an absorbent article. The precursor sheet can be any sheet material that allows a colored region to be readily seen from a body-facing surface of an absorbent article, and can be manufactured from a wide range of materials such as woven, nonwoven materials, latex or thermally bonded airlaid materials, polymeric materials such as apertured formed thermoplastic films, apertured plastic film, hydro-formed thermoplastic films, porous foams, reticulated foams, reticulated thermoplastic films and thermoplastic scrims.

In the present disclosure, a precursor backsheet can be backsheet materials commonly used for absorbent articles such as polyolefinic films like polyethylene, polypropylene and a combination thereof. In some embodiments, the backsheet may be impervious to malodorous gases generated by absorbed bodily discharges, so that the malodors do not escape. The backsheet may or may not be breathable.

The absorbent articles of the present disclosure may further comprise an absorbent core joined with the topsheet, the backsheet, or both in any manner as is known by attachment means such as those well known in the art. Embodiments of the present disclosure are envisioned wherein portions of the entire absorbent core are unattached to either of the topsheet, the secondary topsheet, the backsheet, or more than one of these layers. The absorbent core can be formed from any of the materials well known to those of ordinary skill in the art. Examples of such materials include multiple plies of creped cellulose wadding, fluffed cellulose fibers, wood pulp fibers also known as airfelt, textile fibers, a blend of fibers, a mass or batt of fibers, airlaid webs of fibers, a web of polymeric fibers, and a blend of polymeric fibers. Other suitable absorbent core materials include absorbent foams such as polyurethane foams or high internal phase emulsion (“HIPE”) foams. Suitable HIPE foams are disclosed in U.S. Pat. Nos. 5,550,167, 5,387,207, 5,352,711, and 5,331,015. The absorbent core can comprise superabsorbent materials such as absorbent gelling materials (AGM), including AGM fibers, as is known in the art.

The absorbent articles of the present disclosure may have a pair of flaps on longitudinal sides of a body-facing surface for folding around and securing the absorbent article to the undergarment, the flaps comprising the polymer film and the precursor backsheet. Alternatively, the flaps can comprise the polymer film, nonwoven, and the precursor backsheet. The layers in the flaps can be laminated by either adhesive or thermally bonded means, where thermal bonding includes but is not restricted to technologies such as ultrasonic bonding, cold pressure bonding, and hot pressure bonding. The flaps may have a plurality of third discrete features thereon. The third discrete features may be the same features as one of the first and the second discrete features, or different features from the first and the second discrete features. The plurality of third discrete features can be formed simultaneously with at least one of the plurality of first discrete features and the plurality of second discrete features.

The disclosure is applicable to the production of absorbent articles from discrete components, and it is particularly advantageous for the production of absorbent articles from at least one continuous sheet or web that undergoes processing in a manufacturing process that interacts with the at least one continuous sheet or web to effect a change to the at least one continuous sheet or web. By way of non-limiting examples, the manufacturing process can provide for the conjoining of at least two continuous sheets or web in a nip, the heating of at least one continuous sheet or web, the embossing of at least one continuous sheet or web, the puncturing of at least one continuous sheet or web, the application of fluids to the at least one continuous sheet or web, combinations thereof, and the like.

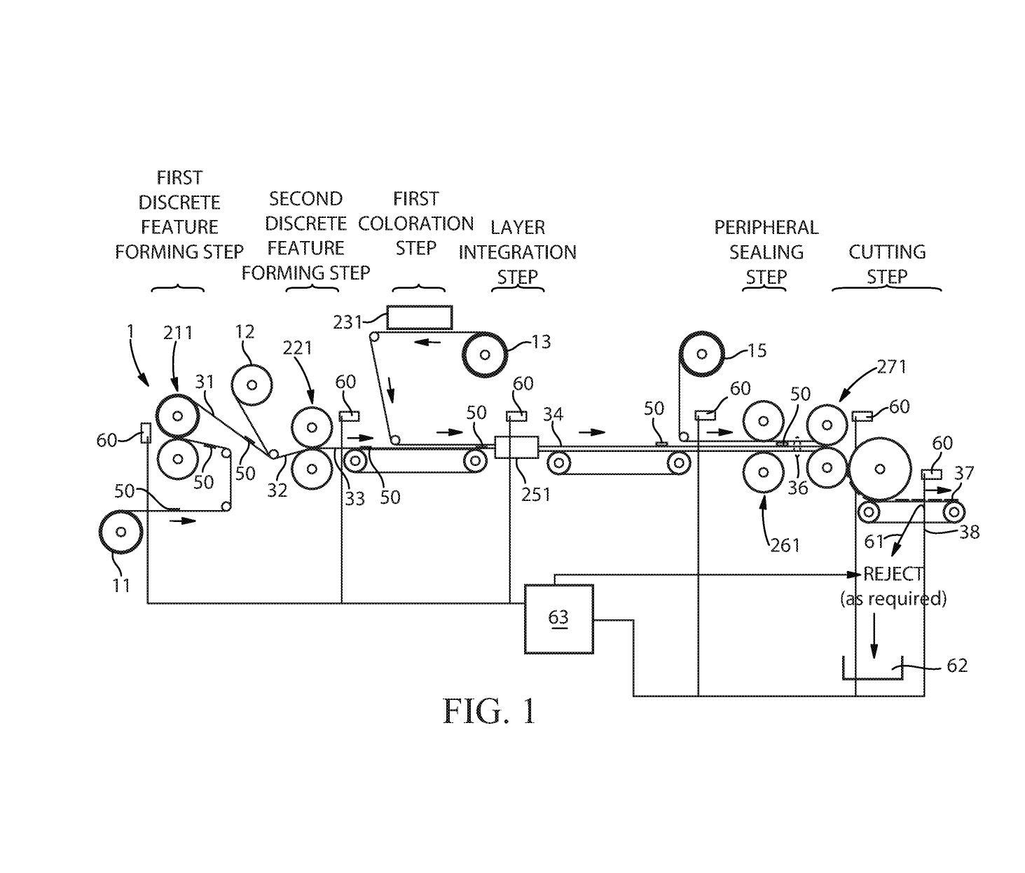

A schematic representing a method and equipment 1 for manufacturing individual absorbent articles 37 according to the present disclosure is depicted in FIG. 1. In FIG. 1, the machine direction is from left to right. The process of the present disclosure may form the absorbent article in an upside down orientation. Alternatively, an absorbent article can be formed top-side up.

An exemplary, but non-limiting, process carried out according to the example in FIG. 1 comprises supplying a polymer film 11 to a first discrete feature forming unit 211 to form a deformed polymer film 31. A plurality of measurement device(s) 50 (also referred to herein as measuring device(s) 50) can be disposed upon (i.e., adhesively, permanently, and/or removeably attached) polymer film 11. Measurement devices 50, their incorporation onto and/or into a polymer film 11, individual absorbent articles 37, and their usefulness will be discussed infra.

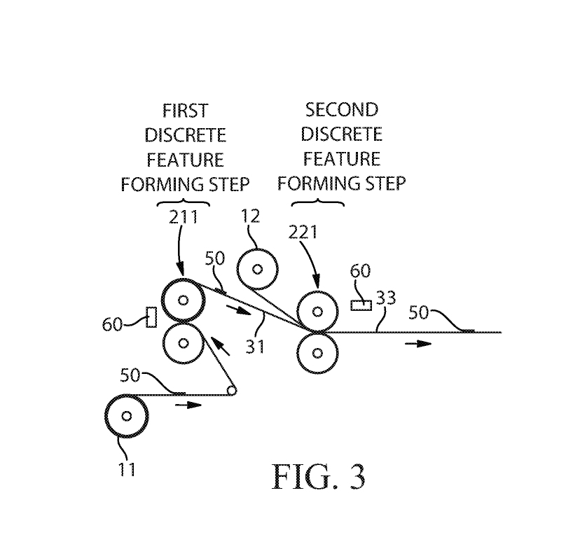

A nonwoven 12 material is then placed in contacting engagement with the deformed polymer film 31 to form a layered composite 32 of the deformed polymer film 31 and the nonwoven 12. The layered composite 32 is then provided to a second discrete feature forming unit 221 to form a deformed layered composite 33. As provided in this example, the first and second discrete feature forming units 211 and 221 may respectively comprise two generally cylindrical rollers where at least one of the two rollers in each discrete feature forming units 211 and 221 have discrete feature forming elements disposed upon its surface. While the process shown in FIG. 1 indicates that the step of layered composite formation and the step of second discrete features formation are carried out sequentially, one of skill in the art will appreciate that these two steps can be carried out simultaneously. This simultaneous process variation is shown in FIG. 3.

Separately, a precursor sheet 13 can be provided with a first colored region in a first coloration unit 231. The colorized precursor sheet 13 can then be placed into contacting engagement with the nonwoven side of the deformed layered composite 33. If the precursor sheet 13 is provided with a colored region before conducting the first coloration step first coloration unit 231, the first coloration step with first coloration unit 231 may be skipped. Alternatively, first coloration unit 231 can provide an additional colored region on the pre-colored precursor sheet 13.

Precursor sheet 13 and the deformed layered composite 33 can be integrated to form an integrated layered composite 34 with an integration unit 251. A precursor backsheet 15 can then be supplied into contacting engagement with a precursor sheet side of the integrated layered composite 34 and integrated by peripheral sealing along a peripheral line of an absorbent article in a peripheral seal unit 261 to form an absorbent article assembly 36. The absorbent article assembly 36 (in continuous web material form) can then be cut in a cutting unit 271 into discrete individual absorbent articles 37.

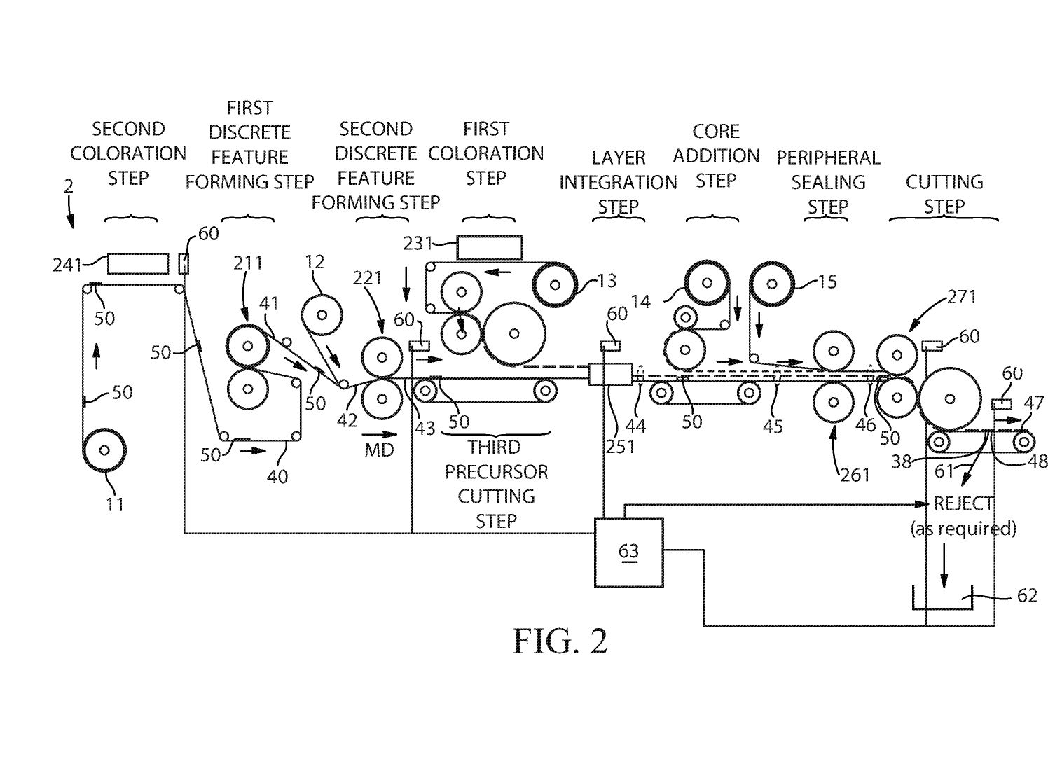

FIG. 2 provides another exemplary, but non-limiting, method and equipment 2 for manufacturing individual absorbent articles 47 according to the present disclosure. The exemplary process provided in FIG. 2 can include several optional steps.

A plurality of measurement device(s) 50 (also referred to herein as measuring device(s) 50) can be disposed upon (i.e., adhesively, permanently, and/or removeably attached) polymer film 11. Measurement devices 50, their incorporation onto and/or into a polymer film 11, individual absorbent articles 37, and their usefulness will be discussed infra.

In the exemplary process shown, a polymer film 11 can be produced with a second colored region in a second coloration unit 241. A plurality of measurement device(s) 50 can be disposed upon polymer film 11. The second colored region can be provided on either side of the polymer film 11. Alternatively, the second colored region can be provided in a nonwoven 12 material. When the polymer film 11 or the nonwoven 12 material is provided with a pre-printed second colored region before conducting the second coloration step with second coloration unit 241, the second coloration step using second coloration unit 241 may be skipped, or still employed to provide additional colored region(s) upon the polymer film 11 or the nonwoven 12 material.

A colored polymer film 40 can be fed into a first discrete feature forming unit 211 to form a deformed polymer film 41. Then, the nonwoven 12 can then be placed into face-to-face contacting engagement with the deformed polymer film 41 to form a layered composite 42. The layered composite 42 can then be fed into a second discrete feature forming unit 221 to form a deformed layered composite 43 material. As shown, the first and second discrete feature forming units 211 and 221 may each comprise two generally cylindrical rollers wherein at least one of the two rollers in each unit has discrete feature forming elements disposed upon its surface.

The steps of forming the layered composite 42 and forming the second discrete features can be carried out sequentially as illustrated in FIG. 2. However, one of skill in the art will appreciate that these steps can be conducted simultaneously as shown in FIG. 3.

The precursor sheet 13 can then be applied onto a nonwoven side of the deformed layered composite 43 in face-to-face contacting engagement. The precursor sheet 13, before being supplied onto the deformed layered composite 43 to form an integrated layered composite 44, can be provided with a first colored region in a first coloration unit 231 and may be cut into a predetermined size and shape and then supplied into face-to-face contacting engagement with a nonwoven side of the deformed layered composite 43. When the precursor sheet 13 already has a first colored region before conducting the first coloration step, the first coloration step may be skipped, or still employed to provide additional colored region on the precursor sheet 13. Then, the deformed layered composite 43 and the precursor sheet 13 can then be integrated to form an integrated layered composite 44.

An absorbent core 14 (which can be provided as a continuous sheet or in a pre-determined size and shape) can be supplied into contacting engagement with a precursor sheet side of the integrated layered composite 44 to form an absorbent layered composite 45 with integration unit 251. A precursor backsheet 15 can be supplied and adhered onto an absorbent core side of the absorbent layered composite 45 to provide peripheral seal in a peripheral seal unit 261 along a peripheral line of an absorbent article and to form an absorbent article assembly 46. The absorbent article assembly 46 is then cut by a cutting unit 271 into individual absorbent articles 47.

### Formation of First and Second Discrete Features

As provided in the present disclosure, the first discrete features and the second discrete features may be of any suitable configuration. Suitable configurations for the features include, but are not limited to: apertures; ridges (continuous protrusions) and grooves (continuous depressions); tufts; columnar shapes; dome-shapes, tent-shapes, volcano-shapes; features having plan view configurations including circular, oval, hour-glass shaped, star shaped, polygonal, polygonal with rounded corners, and the like, and combinations thereof. Polygonal shapes include, but are not limited to rectangular (inclusive of square), triangular, hexagonal, or trapezoidal. In one embodiment, the first discrete features are features selected from the group consisting of apertures, protrusions, depressions, tufts, and combinations thereof, and the second discrete feature are features selected from the group consisting of apertures, protrusions, depressions, tufts, and combinations thereof. In another embodiment, the first discrete features are apertures and the second features are tufts.

The first discrete features and the second discrete features may differ from each other in terms of one or more of the following properties: type, shape, size, aspect ratio, edge-to-edge spacing, height or depth, density, color, surface treatment (e.g., lotion, etc.), number of web layers within the features, and orientation (protruding from different sides of the web). The term “type”, as used herein, refers to whether the feature is an aperture, a protrusion such as a tuft and other kind of protrusion, or a depression.

In the present disclosure, discrete features may be of any suitable size. Typically, either the first features or the second features will be macroscopic. In some embodiments, the first features and the second features will both be macroscopic. The plan view area of the individual features may, in some embodiments of the web, be greater than or equal to about 0.5 mm², 1 mm², 5 mm², 10 mm², or 15 mm², or lie in any range between two of these numbers. The methods described herein can, however, be used to create first features and/or second features that are microscopic which have plan view areas less than 0.5 mm².

Various methods and apparatuses for deforming webs by forming discrete features on webs known in the art can be utilized to form the first and the second discrete features in the present application. Suitable methods are disclosed in U.S. Pat. Nos. 4,189,344; 4,276,336; 4,609,518; 5,143,679; 5,562,645; 5,743,999; 5,779,965; 5,998,696; 6,332,955; 6,739,024; 6,916,969; 7,147,453; 7,423,003; 7,323,072; 7,521,588; U.S. Patent Application Publication Nos. 2004/0110442 A1; 2006/0151914 A1; 2006/0063454 A1; 2007/0029694 A1; 2008/0224351 A1; 2009/0026651 A1; 2010/0201024 A1; European Patent No. EP 1440197 B1; and International Patent Publication Nos. WO2012/148980; WO2012/149074; WO2012/148935; and WO2012/148946.

One type of feature preferred for at least one of the first and the second discrete features in the present disclosure can include apertures. Apertures in a topsheet in an absorbent article may enhance penetration of body exudates through the topsheet into the underlying secondary topsheet or absorbent core. Various methods and apparatii for forming apertures are disclosed in U.S. Pat. Nos. 8,241,543, 3,355,974, 2,748,863, and 4,272,473 (aperture forming methods using apparatus having heated aperture forming elements); U.S. Pat. No. 5,628,097 (method for selectively aperturing a nonwoven web or laminate of a nonwoven web and a polymeric film by weakening the web or the laminate at a plurality of locations); U.S. Pat. No. 5,735,984 (ultrasonic aperturing); U.S. Pat. Nos. 4,342,314 and 4,463,045 (vacuum aperturing); U.S. Pat. Nos. 4,609,518, 4,629,643, and 4,695,422 (hydroforming apertures).

One type of discrete features preferred for the second discrete features in the present disclosure, and would be appreciated by one of skill in the art, can include tufts. In many applications, it can be desirable that fibrous webs have a bulky texture and/or softness. Layered composites in which nonwoven fibers protrude or are partially exposed through a polymer film can be useful as a topsheet in absorbent articles as they provide an absorbent structure in which the nonwoven acts as the conveyor of fluid from one side of the polymer film to the other. The layered composite can be structured such that the fluid collecting side of the layered composite is the polymer film and nonwoven fibers protrude or are partially exposed through the polymer film to the fluid collecting side of the layered composite.

Various methods and apparatii for forming tufts disclosed in patent literature. Exemplary methods are provided in U.S. Pat. Nos. 3,485,706, 4,465,726, and 4,379,799 (forming tufts using a waterjet); U.S. Pat. No. 4,741,941 (forming tufts using air drawing); U.S. Pat. No. 5,080,951 (needle punching); and International Patent Publication Nos. WO 1994/058117, WO 2004/59061, and WO 2010/117636 (method for making tufts on a web using an apparatus comprising a roller comprising a plurality of ridges and grooves).

In one embodiment, the first discrete features are formed by feeding a polymer film in a machine direction into a first nip that is formed between two generally cylindrical rollers, the two rollers having surfaces wherein at least one of the two rollers has first discrete feature forming elements on its surface, and when the polymer film is fed into the nip, the polymer film is deformed.

In another embodiment, the second discrete features are formed by feeding a layered composite of a polymer film and a nonwoven in a machine direction into a second nip that is formed between two generally cylindrical rollers, the two rollers having surfaces wherein at least one of the two rollers has second discrete feature forming elements on its surface, and when the layered composite is fed into the nip, the layered composite is deformed. The two rollers forming the first discrete features and the two rollers forming the second discrete features can be separate pairs of rollers.

The rollers used in the apparatuses and methods described herein are typically generally cylindrical. The term “generally cylindrical”, as used herein, encompasses rolls that are not only perfectly cylindrical, but also cylindrical rollers that may have elements on their surface. The term “generally cylindrical” also includes rollers that may have a step-down in diameter, such as on the surface of the roller near the ends of the roller. This can enable forming deformed elements of different heights in respective zones of the same roller. The rollers are also typically rigid (that is, substantially non-deformable).

An exemplary, but non-limiting, mechanical deformation processes for forming discrete features can utilize a single nip with two rollers comprising discrete male forming elements wherein at least one roller comprises two or more raised ridges, and another approach comprising a multi-hit (multi-nip) configuration that enables controlled placement and orientation of multiple sets of features. Each of these approaches may enable independent control of the features formed in a multi-layer structure, providing additional control over the function and aesthetics of the features. For example, this process could provide the ability to create multi-layer structures where some features have more layers through their thickness than other features.

In one embodiment, the first discrete feature forming elements or the second discrete feature forming elements can be heated.

In one embodiment when a plurality of the first and/or second discrete features are apertures, the first and/or the second discrete feature forming elements can comprise rounded teeth or triangular shaped teeth. Alternatively, in the embodiment, the first and/or the second discrete feature forming elements can comprise being tapered from a base and a tip wherein the base of each tooth has a cross-sectional length dimension greater than a cross-sectional width dimension, wherein each tooth is oriented such that the cross-sectional length dimension of the tooth is disposed at an angle greater than zero relative to a predominant molecular orientation of the polymer film.

In another embodiment, the polymer film may be ring rolled with intermeshing rolls prior to the first discrete feature forming step. Ring-rolling of the film prior to forming the first discrete features, especially apertures, is expected to result in an increase in the size of the apertures and increase in the air permeability of the film.

Referring again to FIGS. 1-2, in another embodiment, the deformed layered composite 33 or 43 may be ring rolled with intermeshing rolls after forming the second discrete features, especially after forming apertures, to spread the apertures apart after formation.

In another embodiment when a plurality of second discrete features are tufts, the second discrete feature forming elements can comprise a plurality of ridges and corresponding grooves which extend unbroken about the entire circumference of a roller which has the second discrete feature forming elements. The tufts may comprise a plurality of tufted fibers being integral extensions of the nonwoven and extending through the polymeric film. In another embodiment when a plurality of second discrete features are tufts, at least part of the distal portion of each of the tufts is covered by a cap, each cap being an integral extension of the polymer film extending over the distal portion of a discrete tuft.

### Provision of a Colored Region on Colored Sheet

In the present disclosure, a first colored region on a precursor sheet can be provided by various methods and apparatus well known to those skilled in the art such as lithographic, screen printing, flexographic, gravure ink jet printing techniques or a method of producing color change using an activatable colorant, and virtually any graphic in any color or color combination can be rendered on the precursor sheet.

### Supplying and Integration of Precursor Sheet

A precursor sheet having a first colored region is introduced by an apparatus, such as a roller, onto the nonwoven side of the deformed layered composite of a polymer film and a nonwoven where first and second discrete features are formed, and the precursor sheet and the deformed layered composite are both moving in a machine direction. The precursor sheet can be introduced to the deformed layered composite as either a continuous layer or a discrete sheet cut before supplied onto the deformed layered composite into a predetermined size and shape. A discrete sheet of the precursor sheet having a first colored region can be prepared by printing a plurality of first colored regions on a continuous liquid pervious precursor sheet and cutting the continuous liquid pervious precursor sheet into discrete sheets in a predetermined shape and size using a cutting means well known to those skilled in the art. The exact dimensions of the size and shape of the discrete sheets may be determined depending on type of an absorbent article. In one example, the colored sheet comprising a precursor sheet is in size and shape which is shorter in length than the final length of an absorbent article such that fluid cannot be transported or wicked to the end of the article. In another embodiment, the colored sheet comprising a precursor sheet extends to the periphery of the topsheet so that the precursor sheet layer underlies the topsheet on the entire inner surface of the topsheet. In the method according to the present disclosure, a precursor sheet and a layered composite of the first and nonwovens can be integrated by various methods and apparatus known to those skilled in the art. For examples, the integration can be carried out by a process selected from the group consisting of cold pressure bonding, heated pressure bonding, ultrasonic bonding, gluing and combinations thereof. Exemplary methods are disclosed in U.S. Pat. Nos. 4,854,984 and 4,919,738 (cold pressure bonding).

In one exemplary, but non-limiting, embodiment, a precursor sheet having a first colored region and a layered composite of a polymer film and a nonwoven can be integrated by heated pressure bonding method comprising forwarding a layered composite of the precursor sheet and the layered composite through a generally cylindrical pattern defining roll and a mating anvil roll. The generally cylindrical pattern defining roll and mating anvil roll may be rotated at matched speeds or at differing speeds to one another.

### Supplying and Integrating Precursor Backsheet

A precursor backsheet can be supplied by an apparatus such as an adhesive bonding roller or thermal sealing roller onto a precursor sheet side of the integrated layered composite 34, 44 which are moving in a machine direction to form an absorbent article assembly 36, 46. When an absorbent core is optionally provided onto the integrated layered composite, the precursor backsheet can be introduced onto an absorbent core side of the absorbent layered composite 45.

The precursor backsheet is integrated to the integrated layered composite so that in an absorbent article a backsheet is preferentially peripherally joined with a topsheet using known techniques, either entirely so that the entire perimeter of the sanitary article is circumscribed by such joinder or are partially peripherally joined at the perimeter.

### Supplying and Integrating Precursor Backsheet

The absorbent article assembly is severed or cut using by a cutting unit conventional in the technical area of absorbent article fabrication into individual absorbent articles to have a predetermined size and shape.

### Measurement/Reading Devices

The measurement devices 50 and an associated reading device 60 (also referred to herein as receiver 60) (the receiver 60 being efficaciously disposed about the absorbent article converting process) are preferably configured to measure or monitor any physical characteristics of the web materials during the various stages of the manufacture of absorbent articles. The measurement devices 50 can be attached to a single web material to monitor progress through a manufacturing process. Alternatively, the measurement devices 50 can be attached to a single web material that is ultimately mated to another web material to monitor process conditions of the web material before and after engagement. Alternatively, the measurement devices 50 can be provided in-between facially mated web materials to monitor the progress of such facially mated web materials through a manufacturing process. Yet still, measurement devices 50 can be positioned upon discontinuous patches of web materials that are then matingly engaged to a web material.

The measurement devices 50 may also be configured to measure and monitor physical characteristics for controlling and monitoring the absorbent article manufacturing process. The characteristics that can be measured can include, e.g. web material temperature, webmaterial/combined web materials deformation (e.g., tension, compression, bending moment, stress, and/or strain), web material and/or process pressure, process forces, web material acceleration (vibration), moisture, speed, pH, residual moisture, residual solvents (e.g., from fluid applications such as inks), micro-point pressures (e.g., pressures from particles that may be in the product, and the like. The measurement devices 50 may transmit measurement data when proximate to the receiver 60, which may further communicate any measurement data to a control unit and/or a data acquisition system capable of processing and/or storing such measurement data. The measurement devices 50 may comprise a transmitter or a transceiver for communicating the measurement data wirelessly to a receiver 60. The measurement devices 50 may be remotely-read untouchably by receiver 60 by means of electromagnetic radiation. Depending on the wavelength, the electromagnetic radiation used can include: radio waves, microwaves, infrared radiation, light, ultraviolet radiation, X-ray radiation, gamma radiation, and the like. Exemplary and suitable measurement devices can include those developed by the Wireless Identification and Sensing Platform of the University of Washington. Suitable reading devices 60 are the model S9028PCL UHF receiver manufactured by Laird Technologies.

It is believed that a suitable measurement device 50 can be capable for the measurement and storage of at least 1000 individual high frequency temperature data points and capable of withstanding operating temperatures above the process maximum operating temperature (e.g., at least about 150° C.) for a desired minimum time (e.g., 1 minute) at a sampling rate of greater than 100,000 HZ for in-nip measurements (e.g., pressure, reject validation, etc.) and 60-1000 Hz for temperatures. Additionally, a suitable measurement device 50 would be capable of meeting a defined maximum continuous operating/storage temperature (e.g., less than 100° C.) and a defined minimum continuous operating/storage temperature (e.g., above 0° C.) as well as being protected from specified external environmental influences (e.g. application of hot conducting liquid). It may also be desirable to provide a suitable measurement device 50 with a reasonable powered-on shelf life (e.g., in the order of months). This is because without external connections the measurement device 50 can be shipped in an always-on standby/sleep state.

Additionally, measurement devices 50 can be provided as microelectromechanical (MEMS), nanoelectromechanical (NEMS) systems, combinations thereof, and the like. Both MEMS and NEMS can be formed from graphene, at least in part, although other materials may be used alternatively as would be understood by those of skill in the art. As would be understood by one of skill in the art, graphene is a single atomic layer of carbon and is the strongest material known to man (where strength is not to be confused with hardness). It also has electrical properties superior to the silicon used to make the chips found in modern electronics. The combination of these properties can make graphene an ideal material for nanoelectromechanical systems, which are scaled-down versions of microelectromechanical systems used for sensing any physical characteristics and any physical phenomena including but not limited to temperature, vibration, and acceleration experienced by web material during the manufacture of absorbent articles as disclosed and envisioned in a manner consistent with the disclosure provided herein.

Due to the continuous shrinking of electrical circuits, particularly those involved in creating and processing radio-frequency signals, they are harder to miniaturize. These ‘off-chip’ components can take up a lot of space and electrical power in comparison to the overall size of ultra-small systems. In addition, most of these radio wave-related components cannot be easily tuned in frequency, requiring multiple copies to ensure the range of frequencies used for wireless communication is covered. Graphene NEMS can address both problems in that they are compact and easily integrated with other types of electronics. Further, their frequency can be tuned over a wide range of frequencies because of the tremendous mechanical strength of graphene.

The measurement devices 50 may also comprise identification information, such as a code, an ID number, or the like. In addition to identification information, measurement devices 50 may comprise at least one other piece of information, which can include web material/absorbent article type number, manufacturer information, order information, date, order number or any other information that can be utilized during the installation, use, maintenance, manufacture, or quality control of the absorbent article or for ordering new web materials. The measurement devices 50 may comprise at least one memory wherein, in addition to the identification information, at least one piece of additional information (such as any physical characteristics of web material/absorbent article measured during use) may be stored. The information stored in the memory can be changed during the process, during repair or installation/removal of a web material, as well as during storage thereof.

The data obtained from the measurement devices 50 may be utilized in controlling the absorbent article converting/manufacturing process, choosing an appropriate web material for an absorbent article converting/manufacturing process, clearing failures during the manufacture of absorbent articles, clearing failures during the processing of web materials, as well as in choosing absorbent article process converting/manufacturing operating parameters. Such an enhanced data acquisition system may thus significantly improve the efficiency and efficacy of the absorbent article process converting/manufacturing process as well as the web materials themselves. Collected data can be forwarded from the data acquisition system for managing the production of, the use of, and/or the storage of the web materials as well as monitoring any necessary absorbent article process converting/manufacturing conditions during the production of absorbent articles.

The measurement device 50 may comprise a tag responding to radio-frequency electromagnetic radiation. Identification distances and wave transmittivity, for instance, may be influenced by using different radio frequencies. The data acquisition system may further utilize tags responding to different frequencies of different sensors that can be used for measurement devices 50 (e.g., temperature, web material deformation, web material/absorbent article and/or process pressure, and the like). Additionally, the measurement devices 50 may comprise a tag, a transponder containing an antenna for receiving radio-frequency electromagnetic radiation as well as a microchip wherein the identification information is stored. Further, the measurement devices 50 may comprise a so-called Radio Frequency Identification (RFID) tag. The tag can be extremely small thereby making it easier to position within or upon the web material(s). Such RFID tags are inexpensive, reliable, and highly available.

Measurement device 50 can be a passive RFID tag which comprises no power source of its own but the extremely low electric current required by its operation is induced by radio-frequency scanning received by the antenna contained within measurement device 50 and transmitted by the receiver 60. By means of this induced current, the tag is able to transmit a response to an inquiry sent by the reading device. In other words, the reading device searches through (e.g., scans) the environment for a tag, and the tag transmits, for example, a measured physical characteristic of papermaking belt 10, any ID code, and/or any other relevant and/or necessary information stored in the microchip (response) after the scanning has induced thereto the electric current necessary for the transmission. The RFID tag may be read at a radio frequency without visual communication and it may be read even through obstacles. In addition, exemplary RFID readers can read a plurality of measurement devices 50, such as RFID tags, simultaneously.

The measurement devices 50 may comprise one or more portable electronic terminal devices suitable as a reading device 60. The reading device 60 may be a data acquisition device, portable computer, palmtop computer, mobile telephone or another electronic device provided with the necessary means for remote-reading a tag. The reading device 60 may comprise a control unit included in the monitoring system. The measurement device 50 and reading device 60 may communicate in either a wired or wireless configuration (e.g., Bluetooth and/or Bluetooth low energy) or any other forms of communication between remote devices such as measurement device 50 and reading device 60 as would be understood by one of skill in the art of wired and wireless communications.

By way of non-limiting example, measurement devices 50 can comprise thermocouples for measuring the temperature of a web material(s)/absorbent article as web material(s)/absorbent article progress through a manufacturing and/or converting process.
Alternatively, the measurement device 50 could comprise a strain gauge sensor that would be suitable for measuring the bending moment, tension, stress, and/or strain present within a given web material(s). Yet still, measurement device 50 could be provided as a pressure sensor, a pH sensor, or even a wear (i.e., erosion) gauge.

The measurement device 50 can be provided as a device responsive to thermal stimuli (e.g., thermocouple). By way of non-limiting example, a thermocouple suitable for use as a measurement device 50 could be woven into the web material(s) used for manufacturing an absorbent article. Alternatively, the measurement device 50 could be disposed upon, and into contacting engagement with, the web material(s) and/or affixed to the web material(s) by needlework or by way of adhesive. Further, measurement device 50 could be printed onto the web material(s) using 3D-printing technology, for example. In any regard, it is preferred that measuring device 50 does not have any adverse impact on the overall desired physical properties of the web material(s)/absorbent article.

If measurement device 50 is provided as a pressure sensor (i.e., a pressure sensor suitable for measuring compressionary forces) it may be preferable for the accompanying electronics to have an overall thickness of less than 0.7 mm. It is believed that such a thickness restraint can provide a pressure sensing measurement device 50 that would be capable of passage through high pressure process nips and pass around any required process idlers (such as those outside of a defined bonding/cutting zone). By way of example, one of skill in the art will recognize that a suitable measurement device 50, provided as a pressure sensor, would provide a sensor that is less than 40 microns thick, capable of withstanding at least about 160,000 psi of pressure, be flexible enough to enable bending around 1″ diameter idler travelling at 10 m/s, as well as be wireless and capable of collecting 10 million points of data at a sample rate of greater than 100 kHz.

Alternatively, it is believed that measurement device 50 can be provided as a portion of a bi-component filament material utilized to form an absorbent article. In other words, the measurement device 50 can be arranged as a filament that includes the measurement device 50 (and any associated electronics) as either the inner or outer portion of a coaxially formed bi-component filament or any other type of high performance tow fiber. In this manner, one of skill in the art will recognize that any number of measurement devices 50 can be woven into and incorporated as part of web material and/or absorbent materials at any location, or in any number of locations, within the confines of the web material and/or absorbent materials used to manufacture an absorbent article.

Yet still, if measurement device 50 is provided as a MEMS or NEMS (discussed supra), it is believed that one of skill in the art could incorporate such a MEMS or NEMS sensor(s) into an adhesive that may be used to bond the web materials used to form an absorbent article. In this way a significant number of measurement devices 50 can be incorporated across a given web material in the CD, over its length in the MD, and combinations thereof. Measurement devices 50 can be disposed collinearly, sinusoidally, randomly, or in any fashion across the CD, MD, and combinations thereof. The use of such MEMS and/or NEMS sensors can significantly reduce any effects and/or impact of disposing a measurement device 50 onto a web material by reducing the amount of physical effort necessary to incorporate a measurement device 50 into the web materials used to form an absorbent article. In other words, the measurement device 50 (and any associated electronics) can be incorporated at any location, or in any number of locations, within the confines of an absorbent article or in any of the precursor portions used to form an absorbent article.

### Reject Validation Sensor

As shown in FIGS. 1-2, it is believed that each respective reading device 60 is operably and electromagnetically connected to each measurement device 50 disposed within and/or upon each web material 11 forming individual absorbent articles 37, 47. A measurement obtained by each measurement device 50 and relayed to a respective reading device 60 can be analyzed to determine whether the individual absorbent article 37 either: 1. Should be rejected because the measurement device 50 detected a characteristic of the individual absorbent articles 37 that falls outside a particular range of desired/required physical properties, or 2. A particular unit operation used to form the individual absorbent article 37 is operating outside a particular range of desired/required process parameters thereby causing the manufactured individual absorbent articles 37 to likely have characteristics that may be outside a particular range of desired/required physical properties and/or consumer-related/desired characteristics.

As discussed in more detail below, defective articles 38, 48 may be subject to a rejection (reject) system 61 and removed from the process. Referring again to FIGS. 1-2, defective articles 38, 48 can be channeled to a reject bin 62. Individual absorbent articles 37, 47 that are not deemed to be defective articles 38, 48 may be subject to further processing steps, such as folding and packaging.

It is to be appreciated that various types of reject systems 61 may be used to physically remove defective articles 38, 48. For example, in some embodiments, a pneumatic system may be used to remove defective absorbent articles 38, 48 from the manufacturing assembly line. More particularly, after application of the final knife and before being folded by a folding mechanism, defective articles 38, 48 could be removed from the assembly line by a blast of compressed air discharged from the pneumatic system. In other embodiments, defective articles 38, 48 may be allowed to advance from the final knife, partially through a folding mechanism, and into a reject bin 62. Such a system could stop or slow the motion of tucker blades on the folding mechanism such that a rejected article 38, 48 will pass through a portion of the folding mechanism without being folded and fall into a reject bin 62. After the defective articles 38, 48 have passed through the folding mechanism, motion of the tucker blades is resumed, allowing the tucker blades to engage non-defective articles and causing the non-defective articles to be folded and channeled toward a packaging process downstream of the folding mechanism.

Referring again to FIGS. 1-2, various measurement devices 50 and other devices may be arranged adjacent the equipment 1 for manufacturing individual absorbent articles 37, 47 may communicate with a controller 63. Based on such communications, the controller 63 may monitor and affect various operations on the converting line 1. As required, the controller 63 may send reject commands to the reject system 61 based on communications with each measurement device 50 and respective reading device 60. In the systems and methods described herein, the controller 63 may include a computer system. The computer system may, for example, include one or more types of programmable logic controller (PLC) and/or personal computer (PC) running software and adapted to communicate on an EthernetIP network. Some embodiments may utilize industrial programmable controllers such as the Siemens S7 series, Rockwell ControlLogix, SLC or PLC 5 series, or Mitsubishi Q series. The aforementioned embodiments may use a personal computer or server running a control algorithm such as Rockwell SoftLogix or National Instruments Labview or may be any other device capable of receiving inputs from sensors, performing calculations based on such inputs and generating control actions through servomotor controls, electrical actuators or electro-pneumatic, electro-hydraulic, and other actuators.

As the substrates and components travel in the machine direction MD through the converting line, the controller tracks the advancement of the substrates (i.e., polymer film 11, non-woven 12, precursor sheet 13, and/or backsheet 15) and/or components (i.e., absorbent core 14) used to manufacture individual absorbent articles 37. In some embodiments such as the exemplary embodiment shown in FIG. 1, the controller 63 may track the advancement with counts generated by a machine axis corresponding with machine direction positions on any substrates and/or components while advancing though the individual absorbent article 37 manufacturing process 1. In some configurations, the machine axis could be configured as an actual motor that provides count signals to the controller 63. The controller 63 could utilize rotational speed, time, and/or count data from the machine axis corresponding with the machine direction speed and travel of the substrates and components through the manufacturing process 10.

As mentioned supra, the systems and methods herein utilize various types of measurement devices 50 to monitor the substrates (i.e., polymer film 11, non-woven 12, precursor sheet 13, and/or backsheet 15) and/or components (i.e., absorbent core 14) used to manufacture individual absorbent articles 37 traveling through the converting line 1. As shown in FIG. 1, various types of measurement devices 50 may be used to detect defects in the substrates (i.e., polymer film 11, non-woven 12, precursor sheet 13, and/or backsheet 15) and/or components (i.e., absorbent core 14). In particular, the measurement devices 50 may detect defects within substrates and/or components themselves, such as for example, damage, holes, tears, dirt, and the like, and may also detect defective assemblies and/or combinations of the substrates and components, such as for example, missing and/or misplaced ears, landing zones, fasteners, and the like. Additionally, as discussed supra, various types of measurement devices 50 may be used to detect defects and/or anomalies in the manufacturing process of individual absorbent articles 37. For example, the measurement devices 50 may detect excessive or insufficient nip pressures, excessive or insufficient process temperatures, excessive or insufficient adhesive application rates, excessive or insufficient web material tensions, excessive or insufficient web material speeds, and the like. It is believed that based on the detections of each measurement devices 50, feedback signals from the measurement devices 50 in the form of inspection parameters can be communicated to the controller 63.

As shown in FIG. 1, each respective reading device 60 can be connected with the controller 63 through a communication network, which allows each measurement device 50 to communicate measurements to the controller 63. As discussed in more detail below, each device that communicates on the network can each include precision clocks that are synchronized to a master clock having some specified and/or desired accuracy. As shown in FIG. 1, each controller 63 may be connected directly with a communication network. As such, each respective reading device 60 connected directly with the communication network may include a clock. Each respective reading device 60 that includes a clock and that may be connected directly with the communication network may include, for example, vision systems such as National Instruments CVS or any PC-based vision system such as Cognex VisionPro. Such sensors may also include other controllers that may be configured as peers to the controller or may be configured as subordinate to the controller.

In some embodiments, each respective reading device 60 may be indirectly connected with the communication network. For example, each respective reading device 60 may be connected with the communication network through a remote input and output (I/O) station. When utilizing remote I/O stations, each respective reading device 60 may be hardwired to the remote I/O stations, and in turn, the remote I/O stations are connected with the communication network. As such, the each remote I/O station may include a precision clock. Example remote I/O stations or other IEEE-1588 based instruments that can be utilized with systems and methods herein include, for example a National Instruments PCI-1588 Interface (IEEE 1588 Precision Time Protocol Synchronization Interface) that synchronizes PXI systems, I/O modules and instrumentation over Ethernet/IP or a Beckhoff Automation EtherCat and XFC technology (eXtreme Fast Control Technology).

In one configuration, the controller 63 can include the master clock, and all other clocks of devices connected with the communication network are referenced to the controller master clock. In such a configuration, the remote I/O stations and inspection sensors each include a clock that is synchronized to the controller master clock. A process parameter measured by each measurement device 50 can be communicated to the communication network from a respective reading device 60, and time-stamped with the time from the clocks, on the corresponding sensors and any remote I/O station. In turn, the inspection parameters and corresponding timestamp data is sent to the controller 63 over the communication network. Thus, the controller 63 can be programmed to evaluate the process parameter measured by each measurement device 50 based on the actual time the inspection parameter was provided by the measurement device 50. Therefore, ambiguity as to when detections were actually made by each measurement device 50 can be small. The controller 63 can then direct the reject system 61 to physically remove defective articles 38, 48 as discussed supra.

### Registration

Absorbent articles comprise multiple functional and/or aesthetic components including compositional elements such as a topsheet, a backsheet and optionally secondary topsheet and an absorbent core, and design elements such as colored regions, discrete features formed on a topsheet, and optionally channels and a logo. During the manufacturing of absorbent articles, the position of components of article in each step of the manufacturing process may affect the overall quality of the articles and the acceptance of the articles by consumers as consumers often desire consistency in the configuration of purchased goods for both functional and aesthetic reasons. For example, locations of the first discrete features, the second discrete feature, the first colored region and/or the second colored region need to be controlled as designed to secure best functional and aesthetic goods. To ensure consistency throughout the manufacturing process, components must be positioned uniformly.

One of skill in the art will appreciate that various methods and systems for inspecting the locations of selected components of an absorbent article during a manufacturing process can be used cooperatively utilized with the apparatus and process of the present disclosure. Suitable inspection systems are disclosed in U.S. Pat. No. 5,359,525; European Patent No. EP 2090 951 A1; and International Patent Publication No. WO 2012/161709 (systems and methods for the automated regulation of production lines).

In a method according to the present disclosure, a phasing can be conducted in between two consecutive steps to determine and control position of at least one component, and may be carried out at least once.

### Formation of a Plurality of Discrete Extended Elements

A method according to the present disclosure optionally comprises a step of forming a plurality of discrete extended elements on either a polymer film, or a layered composite of a polymer film and a nonwoven. Hereinafter in this section of Formation of a Plurality of Discrete Extended Elements, a polymer film, and a layered composite of a polymer film and a nonwoven are collectively denoted as a precursor web. It can be beneficial for the precursor web to have a textured surface by having a plurality of discrete extended elements which can provide the surface of the polymer film with a desirable feel, visual impression, and/or audible impression.

A plurality of discrete extended elements can be made in a vacuum forming process, a hydroforming process, a high static pressure forming process, a solid state deformation process in mated forming structures, or methods using a forming structure and a compliant substrate. With a typical vacuum forming process, a precursor web is heated and placed over a forming structure. Then a vacuum forces the precursor web to conform to the texture of the forming structure. The resulting web has texture that can provide a soft and silky tactile impression, depending upon the texture of the forming structure and degree of conformation. With a typical hydroforming process, a precursor web is placed over a forming structure and high pressure and high temperature water jets force the precursor web to conform to the texture of the forming structure. A suitable high static pressure forming process employs a high pressure gas to deform the precursor web to the texture of a forming structure is disclosed in International Patent Publication Nos. WO 10/105002, WO 10/105017, and WO 11/112213. Solid state deformation processes can convey the web between mating forming structures such as those disclosed in International Patent Publication No. WO 12/148936, or use a compliant substrate to impress the discrete extended elements into the precursor web as disclosed in International Patent Publication Nos. WO 10/105009 and WO 10/105019.

A plurality of discrete extended elements in the present disclosure comprise open proximal ends, open or closed distal ends, and sidewalls, wherein the discrete extended elements comprise thinned portions at the distal ends of the discrete extended elements and/or along the sidewalls of the discrete extended elements, and wherein (a) the discrete extended elements have a diameter of less than about 500 microns, (b) the discrete extended elements have an aspect ratio of at least about 0.2, and/or (c) the polymer film comprises at least about 95 discrete extended elements per square centimeter.

In an embodiment, a plurality of discrete extended elements can be formed by a process comprising the steps of: i) providing a forming structure comprising a plurality of discrete protruded elements and lands completely surrounding the discrete protruded elements; ii) providing a compliant substrate; iii) providing a precursor web between the compliant substrate and the forming structure; and iv) providing pressure between the compliant substrate and the forming structure sufficient to conform the precursor web to the discrete protruded elements of the forming structure to form the embossed web.

In another embodiment, a plurality of discrete extended elements can be formed by a process comprising the steps of: i) feeding a precursor web between a static gas pressure plenum and a forming structure comprising a plurality of discrete protruded elements, the discrete protruded elements having a height of at least substantially equal to a thickness of the precursor web; and ii) applying pressure from the static gas pressure plenum against the precursor web opposite the forming structure creating a pressure differential across the precursor web sufficient to conform the precursor web to the discrete protruded elements of the forming structure.

In another embodiment, a plurality of discrete extended elements can be formed by a process comprising the steps of: i) feeding a precursor web between a static gas pressure plenum and a forming structure comprising a plurality of discrete apertures, discrete depressions, or combinations thereof, the apertures or depressions having a depth of at least substantially equal to a thickness of the precursor web; and ii) applying pressure from the static gas pressure plenum against the precursor web opposite the forming structure creating a pressure differential across the precursor web sufficient to force the precursor web into the apertures or depressions of the forming structure, thereby forming the precursor web comprising a plurality of discrete extended elements.

In another embodiment, a plurality of discrete extended elements can be formed by a process comprising the steps of: i) providing a precursor web, ii) providing a pair of mated forming structures, including a first forming structure and a second forming structure, wherein at least the first forming structure comprises voids, and wherein at least the second forming structure comprises protrusions; and iii) moving the web through a deformation zone between the mated forming structures, wherein the voids of the first forming structure engage with the protrusions of the second forming structure at an engagement position.

### Provision of a Second Colored Region

A method according to the present disclosure optionally comprises a step of providing a second colored region on the polymer film or the nonwoven either prior to or after formation of the first discrete features. The second colored region can be provided either side of the polymer film or nonwoven. Regarding coloration methods, descriptions in the section of Provision of a Colored Region on Colored Sheet above are applicable for provision of the second colored region.

### Introduction of Absorbent Core

A method according to the present disclosure optionally further comprises a step of supplying and integrating an absorbent core to a precursor sheet side of the integrated layered composite 34, 44 moving in a machine direction to form an absorbent layered composite 45. The absorbent core can be supplied as a preformed core. In one embodiment, the absorbent core is cut in a predetermined size and shape before being provided onto the precursor sheet side of the integrated layered composite.

An absorbent core can be integrated to the integrated layered composite by various methods and apparatus known in the art such as cold pressure bonding, heated pressure bonding, ultrasonic bonding, gluing and combinations thereof. In one embodiment, an absorbent core can be integrated to the absorbent layered composite by gluing.

### Application of Lotion Composition

Treatments of an absorbent article with lotion have been proposed to provide skin health benefits and to allow fluid to be absorbed into the article. To provide an absorbent article treated with a lotion, a method of the present disclosure may further comprise a step of applying a lotion composition to at least a portion of a topsheet, the inner surface of the backsheet, and/or any substrate (or surface thereof) disposed between the topsheet and the backsheet such as a secondary topsheet and an absorbent core. The lotion composition can be a liquid, a solid or a semi-solid at room temperature, and comprise at least one skin benefit agent. The lotion composition can be applied in any known manner, in any known pattern, and to any known portion of the absorbent article, as is well known in the art of lotioned absorbent articles. For example, the lotion composition can be applied in a pattern of generally parallel stripes or bands. The lotion composition can be a lotion coating on any part of the article, and on either side of any layer, such as upper surfaces, or lower surfaces. In one embodiment, a lotion composition can be disposed on the inner surface of the topsheet by disposing the lotion composition on at least one of a lower side of a polymer film, an upper side, and a lower side of a nonwoven. The lotion can be applied prior to the first discrete forming step, after the first discrete forming step and/or after the second discrete forming step.

### Application of Odor Control Composition

A method of the present disclosure may further comprise a step of applying an odor control composition. Use of a fragrance composition and/or an odor control composition in absorbent articles has been proposed for controlling and reducing malodors in the articles. In general, suitable components for odor control compositions include reactive components. Reactive components include components that can react with malodors, such as ammonia-based malodors or sulphur-based malodors (i.e. “malodor reactive components”), and components that mask malodors and/or react with receptors of the nose to block the perception of malodor by the nose of a consumer (i.e. “malodor masking components”). Suitable reactive components are described, for example, in U.S. Patent Publication No. 2008/0071238 A1 and International Patent Publication Nos. WO 07/113778 and WO 08/114226.

An odor control composition may be applied on or within a layer of an absorbent article in any known manner, in any known pattern, and to any known portion of the absorbent article, as is well known in the art of absorbent articles. This means that, since the absorbent article is constituted by a series of layers, the odor control composition is applied onto one of the surfaces of these layers. An odor control composition can be applied onto the surface of application with any possible application pattern. In some cases it is possible that the odor control composition is applied on more than one layer within the article.

### Examples

A. A method for fabricating an absorbent article, the absorbent article comprising at least a topsheet and a liquid impermeable backsheet, the method comprising the steps of:
a) supplying the topsheet;
b) supplying the liquid impermeable backsheet;
c) affixing a measuring device to one of the topsheet or liquid impermeable backsheet;
d) forming the absorbent article by contactingly engaging the topsheet and liquid impermeable backsheet so that the measuring device is disposed between the topsheet and liquid impermeable backsheet, when the topsheet and liquid impermeable backsheet are disposed in contacting engagement.
B. The method for fabricating an absorbent article of A further comprising the steps of:
e) providing an absorbent article converter, the absorbent article converter having at least one process set-point, the at least one process set-point being related to at least a first physical characteristic of the absorbent articles;
f) causing the contactingly engaged topsheet and liquid impermeable backsheet to traverse past a receiver, the receiver being in wirelessly communicating engagement with the measuring device when the measuring device is proximate the receiver, the measuring device being capable of wirelessly transmitting information to the receiver, the information comprising data relating to a measurement of the at least one physical characteristic of the contactingly engaged topsheet and liquid impermeable backsheet; and,
g) changing the process set-point according to the measurement of the at least one physical characteristic of the contactingly engaged topsheet and liquid impermeable backsheet.
C. The method of B further comprising the step of collecting the data to form an absorbent article profile.
D. The method of any of A-C further comprising the step of changing the at least one process set-point according to the absorbent article profile.
E. The method of any of A-D further comprising the steps of providing the absorbent article converter with a compressionary process and disposing the receiver proximate to the compressionary process such that the measurement device transmits data relating to compressionary forces observed by the contactingly engaged topsheet and liquid impermeable backsheet while interposed within the compressionary process.
F. The method of any of A-E further comprising the steps of providing the absorbent article converter with a heating process and disposing the receiver proximate to the heating process such that the measurement device transmits data relating to temperatures observed by the contactingly engaged topsheet and liquid impermeable backsheet while disposed within the heating process.
G. The method of any of A-F further comprising the step of providing the at least one physical characteristic of the contactingly engaged topsheet and liquid impermeable backsheet as a physical characteristic selected from the group consisting of temperature, pressure, pH, stress, strain, bending moment, acceleration, and combinations thereof.
H. The method of any of A-G further comprising the step of providing the measuring device as a device responsive to thermal stimuli.
I. The method of any of A-H further comprising the step of providing the measuring device as a pressure sensor.
J. The method of any of A-I further comprising the step of providing the absorbent article converter with a reject system, the reject system being responsive to remove an absorbent article from the absorbent article converter when the measuring device measures the at least one physical characteristic of the contactingly engaged first and second web materials and the at least one physical characteristic of the contactingly engaged first and second web materials measured by the measuring device is not equal to the at least one process set-point.
K. A method for adjusting a process for manufacturing absorbent articles, the process having a machine direction (MD) and a cross-machine direction (CD) coplanar and orthogonal thereto, said process improving the manufacture of absorbent articles manufactured thereby, the process comprising the steps of:
   a) providing an absorbent article converter, the absorbent article converter having at least one process set-point, the at least one process set-point being related to at least a first physical characteristic of the absorbent articles;
   b) providing a first web material associated with the absorbent articles integral with the absorbent article converter;
   c) attaching a measuring device upon a surface of the first web material, the measuring device being disposed integral thereupon;
   d) providing a second web material associated with the absorbent articles integral with said converter;
   e) contactingly engaging the first and second web materials with the converter to provide a contactingly engaged first and second web materials so that the measuring device is disposed between the first and second web materials;
   f) causing the contactingly engaged first and second web materials to traverse past a receiver, the receiver being in wirelessly communicating engagement with the measuring device when the measuring device is proximate the receiver, the measuring device being capable of wirelessly transmitting information to the receiver, the information comprising data relating to a measurement of the at least one physical characteristic of the contactingly engaged first and second web materials; and,
   g) changing the process set-point according to the measurement of the at least one physical characteristic of the contactingly engaged first and second web materials.
L. The method of K further comprising the step of collecting the data to form an absorbent article profile.
M. The method of any of K-L further comprising the step of changing the at least one process set-point according to the absorbent article profile.
N. The method of any of K-M further comprising the steps of providing the absorbent article converter with a compressionary process and disposing the receiver proximate to the compressionary process such that the measurement device transmits data relating to compressionary forces observed by the contactingly engaged first and second web materials while interposed within the compressionary process.
O. The method of any of K-N further comprising the steps of providing the absorbent article converter with a heating process and disposing the receiver proximate to the heating process such that the measurement device transmits data relating to temperatures observed by the contactingly engaged first and second web materials while disposed within the heating process.
P. The method of any of K-O further comprising the step of providing the at least one physical characteristic of the contactingly engaged first and second web materials as a physical characteristic selected from the group consisting of temperature, pressure, pH, stress, strain, bending moment, acceleration, and combinations thereof.
Q. The method of any of K-P further comprising the step of providing the measuring device as a thermocouple.
R. The method of any of K-Q further comprising the step of providing the measuring device as a pressure sensor.
S. The method of any of K-R further comprising the step of providing the absorbent article converter with a reject system, the reject system being responsive to remove an absorbent article from the absorbent article converter when the measuring device measures the at least one physical characteristic of the contactingly engaged first and second web materials and the at least one physical characteristic of the contactingly engaged first and second web materials measured by the measuring device is not equal to the at least one process set-point.
T. A method for the in-situ measurement of at least one physical property of a web material traversing through web material processing equipment, the method comprising the steps of:
a) supplying the web material;
b) affixing a measuring device to the web material;
c) processing the web material with the web material processing equipment; and,
d) causing the web material to traverse past a receiver while the web material is integral with the web material processing equipment, the receiver being in wirelessly communicating engagement with the measuring device when the measuring device is proximate the receiver, the measuring device being capable of wirelessly transmitting information to the receiver, the information comprising data relating to a measurement of the at least one physical characteristic of the web material while the web material is integral with the web material processing equipment.
U. The method of T further comprising the step of changing a process set-point according to
V. The method or any of T-U further comprising the step of providing the web material processing equipment with at least one process set-point, the at least one process set-point being related to at least a first physical characteristic of the web material.
W. The method of any of T-V further comprising the step of collecting the data to form a web material profile.
X. The method of any of T-W further comprising the step of changing the at least one process set-point according to the web material profile.
Y. The method of any of T-X further comprising the steps of providing the web material processing equipment with a compressionary process and disposing the receiver proximate to the compressionary process such that the measurement device transmits data relating to compressionary forces observed by the web material while interposed within the compressionary process.
Z. The method of any of T-Y further comprising the steps of providing the web material processing equipment with a heating process and disposing the receiver proximate to the heating process such that the measurement device transmits data relating to temperatures observed by the web material while disposed within the heating process.
AA. The method of any of T-Z further comprising the step of providing the at least one physical characteristic of the web material as a physical characteristic selected from the group consisting of temperature, pressure, pH, stress, strain, bending moment, acceleration, and combinations thereof.
BB. The method of any of T-AA further comprising the step of providing the measuring device as a device responsive to thermal stimuli.
CC. The method of any of T-BB further comprising the step of providing the measuring device as a pressure sensor.
DD. The method of any of T-CC further comprising the steps of providing a second web material and contactingly engaging the web material and second web material in a face-to-face relationship so that the measuring device is disposed between the web material and second web material.
EE. The method of any of T-DD further comprising the step of forming the web material and second web material into an assembled article.
FF. The method of T-DD further comprising the steps of disposing a third web material between the web material and second web material.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A method for fabricating an absorbent article, the absorbent article comprising at least a topsheet and a liquid impermeable backsheet, the method comprising the steps of:
a) supplying the topsheet;
b) supplying the liquid impermeable backsheet;
c) affixing a measuring device to one of the topsheet and liquid impermeable backsheet;
d) contactingly engaging the topsheet and liquid impermeable backsheet so that the measuring device is disposed therebetween when the topsheet and liquid impermeable backsheet are disposed in contacting engagement.

2. The method for fabricating an absorbent article of claim 1 further comprising the steps of:
e) providing an absorbent article converter, the absorbent article converter having at least one process set-point, the at least one process set-point being related to at least a first physical characteristic of the absorbent articles;
f) causing the contactingly engaged topsheet and liquid impermeable backsheet to traverse past a receiver, the receiver being in wirelessly communicating engagement with the measuring device when the measuring device is proximate the receiver, the measuring device being capable of wirelessly transmitting information to the receiver, the information comprising data relating to a measurement of the at least one physical characteristic of the contactingly engaged topsheet and liquid impermeable backsheet; and,
g) changing the process set-point according to the measurement of the at least one physical characteristic of the contactingly engaged topsheet and liquid impermeable backsheet.

3. The method of claim 2 further comprising the step of collecting the data to form an absorbent article profile.

4. The method of claim 3 further comprising the step of changing the at least one process set-point according to the absorbent article profile.

5. The method of claim 2 further comprising the steps of providing the absorbent article converter with a compressionary process and disposing the receiver proximate to the compressionary process such that the measurement device transmits data relating to compressionary forces observed by the contactingly engaged topsheet and liquid impermeable backsheet while interposed within the compressionary process.

6. The method of claim 2 further comprising the steps of providing the absorbent article converter with a heating process and disposing the receiver proximate to the heating process such that the measurement device transmits data relating to temperatures observed by the contactingly engaged topsheet and liquid impermeable backsheet while disposed within the heating process.

7. The method of claim 2 further comprising the step of providing the at least one physical characteristic of the contactingly engaged topsheet and liquid impermeable backsheet as a physical characteristic selected from the group consisting of temperature, pressure, pH, stress, strain, bending moment, acceleration, and combinations thereof.

8. The method of claim 1 further comprising the step of providing the measuring device as a device responsive to thermal stimuli.

9. The method of claim 1 further comprising the step of providing the measuring device as a pressure sensor.

10. The method of claim 1 further comprising the step of providing the absorbent article converter with a reject system, the reject system being responsive to remove an absorbent article from the absorbent article converter when the measuring device measures the at least one physical characteristic of the contactingly engaged first and second web materials and the at least one physical characteristic of the contactingly engaged first and second web materials measured by the measuring device is not equal to the at least one process set-point.

11. A method for adjusting a process for manufacturing absorbent articles, the process having a machine direction (MD) and a cross-machine direction (CD) coplanar and orthogonal thereto, said process improving the manufacture of absorbent articles manufactured thereby, the process comprising the steps of:
a) providing an absorbent article converter, the absorbent article converter having at least one process set-point, the at least one process set-point being related to at least a first physical characteristic of the absorbent articles;
b) providing a first web material associated with the absorbent articles integral with the absorbent article converter;
c) attaching a measuring device upon a surface of the first web material, the measuring device being disposed integral thereupon;
d) providing a second web material associated with the absorbent articles integral with said converter;
e) contactingly engaging the first and second web materials with the converter to provide a contactingly engaged first and second web materials so that the measuring device is disposed between the first and second web materials;
f) causing the contactingly engaged first and second web materials to traverse past a receiver, the receiver being in wirelessly communicating engagement with the measuring device when the measuring device is proximate the receiver, the measuring device being capable of wirelessly transmitting information to the receiver, the information comprising data relating to a measurement of the at least one physical characteristic of the contactingly engaged first and second web materials; and,
g) changing the process set-point according to the measurement of the at least one physical characteristic of the contactingly engaged first and second web materials.

12. The method of claim 11 further comprising the step of collecting the data to form an absorbent article profile.

13. The method of claim 12 further comprising the step of changing the at least one process set-point according to the absorbent article profile.

14. The method of claim 11 further comprising the steps of providing the absorbent article converter with a compressionary process and disposing the receiver proximate to the compressionary process such that the measurement device transmits data relating to compressionary forces observed by the contactingly engaged first and second web materials while interposed within the compressionary process.

15. The method of claim 11 further comprising the steps of providing the absorbent article converter with a heating process and disposing the receiver proximate to the heating process such that the measurement device transmits data relating to temperatures observed by the contactingly engaged first and second web materials while disposed within the heating process.

16. The method of claim 11 further comprising the step of providing the at least one physical characteristic of the contactingly engaged first and second web materials as a physical characteristic selected from the group consisting of temperature, pressure, pH, stress, strain, bending moment, acceleration, and combinations thereof.

17. The method of claim 11 further comprising the step of providing the measuring device as a thermocouple.

18. The method of claim 11 further comprising the step of providing the measuring device as a pressure sensor.

19. The method of claim 11 further comprising the step of providing the absorbent article converter with a reject system, the reject system being responsive to remove an absorbent article from the absorbent article converter when the measuring device measures the at least one physical characteristic of the contactingly engaged first and second web materials and the at least one physical characteristic of the contactingly engaged first and second web materials measured by the measuring device is not equal to the at least one process set-point.
